(19) United States
(12) Patent Application Publication
(10) Pub. No.: US 2009/0082711 A1
(43) Pub. Date: Mar. 26, 2009

(54) Bandaging structure and methodology

(75) Inventor: Gerhard Paasche, Scappoose, OR (US)

Correspondence Address: ROBERT D. VARITZ, P.C., 4915 SE 33RD PLACE, PORTLAND, OR, 97202, US

(73) Assignee: MJD Innovations, L.L.C.

(21) Appl. No.: 12/313,169

(22) Filed: Nov. 17, 2008

Related U.S. Application Data
(63) Continuation-in-part of application No. 11/983,259, filed on Nov. 7, 2007.
(60) Provisional application No. 61/003,969, filed on Nov. 21, 2007.
(60) Provisional application No. 60/859,770, filed on Nov. 16, 2006.

(51) Int. Cl. A61F 13/00

(52) U.S. Cl. 602/53

(57) ABSTRACT

A dynamic-action, pliable, anatomically conformable bandaging structure for application to an anatomical wound including a pliable, low-rebound, viscoelastic, acceleration-rate-sensitive cushioning expanse which is placeable confrontingly adjacent such a wound, and operatively associated and structurally united with that cushioning expanse, adjustable, pressure-fluid-inflatable structure. An associated methodology features the steps of (a) applying over a wound area a first, dynamic-action bandaging structure in the form of the mentioned cushioning material, (b) by such applying, furnishing dynamic, low-rebound, viscoelastic, acceleration-rate-sensitive pressure-applying behavior, and (c) utilizing the mentioned pressure-fluid-inflatable structure, applying, through the cushioning material, collaborative, dynamic, controlled, contained pressure-fluid action.

### CROSS REFERENCES TO RELATED CASES

This application claims priority to currently copending U.S. Provisional Patent Application Ser. No. 61/003,969, filed Nov. 21, 2007, for Bandaging Structure and Methodology, and is a Continuation-In-Part of currently copending U.S. Regular patent application Ser. No. 11/983,259, filed Nov. 7, 2007, for Bandaging Structure and Methodology which claims priority to U.S. Provisional Patent Application Ser. No. 60/859,770, for Bandaging Structure and Methodology, filed Nov. 16, 2006, which second-mentioned provisional application describes improvements and variations to the subject matter disclosed, illustrated and claimed in U.S. Pat. No. 6,812,375 B2, granted Nov. 2, 2004, for Pressure-Evenizing Low-rebound Wound Dressing. The disclosure contents of the two mentioned provisional patent applications, of the mentioned regular patent application, and of the mentioned patent, are hereby incorporated herein by reference.

### BACKGROUND AND SUMMARY OF THE INVENTION

This invention relates to wound bandaging, and in particular to dynamic-action, pliably, anatomically conformable, wound-bandaging structure, and to an associated methodology, which utilize, among other things, the direct application to the outside anatomical surface area adjacent a wound, internal or external (as from surgery), of a low-rebound, acceleration-rate-sensitive, preferably viscoelastic foam cushioning structure which may be urged pliably against the wound area, and, selectively, by varying amounts of “back-up” bandaging-assisting fluid pressure contained in a fluid plenum which forms an integrated part of the wound-bandaging structure of the invention. The methodology of the invention, among other things, involves the selective application of wound-healing, evenized pressure under the influence of one or both (when desired, and how much) of these two, structurally integrated and cooperative pressure-applying mechanisms.

Those who are skilled in the relevant medical arts involving the healing of wounds, such as the two broadly illustrative types of wounds just mentioned above, are quite familiar with various aspects of the anatomical healing process. The present invention, while not directly, though definitely tangentially, involved with many aspects of the “anatomical” technology of healing, is extremely involved with the application of controlled, pressure-applying bandaging and dressing in such a context which have been proven to promote significantly rapid and extremely successful healing experiences for a variety of anatomical wounds. Bandaging technology which is related, in an interesting background sense, to the technology of the present invention is described in above-identified U.S. Pat. No. 6,812,375, and the reader is encouraged to look at the content in that patent.

In this setting, the present invention relates to a dynamic-action, pliable and anatomically conformable bandaging structure and an associated methodology which enable bandaging of an anatomical wound area, internal or external, whereby the region, or area, which is bandaged and thus “engaged” by the structure of the invention, is subjected to a substantially even and uniform, continuously dynamically adaptable and controllable, overall pressure, without such bandaging producing, or allowing, any appreciable high-pressure or low-pressure “bandaging-contact” points/expanses. A notable feature about the structure and practice of the invention is that maintenance of such wound-bandaging conditions is greatly facilitated and enhanced by the cooperative interaction of two differently structured dynamically behaving mechanisms, one of which is the above-mentioned acceleration-rate-sensitive, etc. cushioning material which is closely proximate a bandaged area, and the other of which is the “back-up” activity of the adjustable, pressure-fluid plenum wherein selected fluid pressure, gas or liquid, tends dramatically to maintain the evenized overall pressure no matter what occurs within the wound-bandaged area per se, such as anatomical motion due to normal muscular and or other anatomical behavior, as well as expected pulsatile behavior of blood flow which takes place in the bandaged area. Anatomically-conforming, overall pliability in the bandaging structure of the invention is one of several important structural and functional departures from the predecessor structure illustrated and described in the incorporated '259 patent application wherein pressure fluid activity is accommodated by a generally rigid back-up structure which lies in operative contact with acceleration-rate-sensitive, effectively “anatomy contacting” cushioning foam material.

It is very well known that, with respect to the healing of various wounds, particularly such as those that result from surgical procedures, it is extremely important that various fluid flows into and through the healing wound area, dictated by normal anatomical behavior, such as blood flow in that area, be allowed to continue substantially normal anatomical-healing behavior, in order to speed the healing process. It is also important, regarding certain instances of anatomical, fluid-flow behavior, to prevent a potentially problematic build-up of pooled, excess fluid in pockets under the skin in the region of a wound dressing.

As those skilled in the medical arts clearly understand, many conventional bandaging approaches do not achieve ideal pressure-applying conditions in a bandaged wound area. Very specifically, it is common, in many prior art practices, that a pressure-applying bandage will not properly, and most desirably, in a most appropriate, pressure-applying manner, actually topographically follow the underlying contours of the anatomy in a wound area.

This kind of situation frequently produces, as one unfortunate result, a bandaging condition which ends up applying unacceptably high levels of pressure to certain wound regions, such as regions overlying a blood vessel. Such elevated pressure will frequently tend to constrict blood flow during a wound-bandaging period, and will thus prolong the healing process. In a worst case scenario, such bandaging may actually introduce additional injury.

Additionally, conventional bandaging often ends up applying significant underpressure in certain wound regions, usually immediately adjacent “overpressure” regions, which underpressure regions can then promote the above-mentioned, unwanted and disturbing pooling of excess fluid (referred to as edema) in these underpressure regions.

Continuing with relevant, general background discussion regarding wound healing and associated bandaging, as substantially all people recognize, surface and immediate sub-surface trauma (wounds) to tissue can be caused through accidents or surgery, and may typically appear with tissue separation and/or as bruising. In all such situations, post traumatic wound care requires thoughtfully administered care. Among major considerations which must be taken into account in terms of promoting wound healing, is recognition that, where tissue has been separated, it must be rejoined and allowed to reunite. Another consideration is that appropriate blood flow must be maintained in a wound area in order to promote the most rapid possible healing, and also to avoid the potential for further injury resulting from poorly managed blood flow.

Those skilled in the medical arts and familiar generally with the wound-healing modalities available for different kinds of wounds, are very familiar with these above, and other, problems, concerns and considerations that need to be taken into account in the realm of wound bandaging. As is suggested above, some of the background materials mentioned above herein describe certain effective predecessor ways of dealing with wound managing in a fashion such that healing time can be shortened, and healing-time injury caused by improper bandaging can largely be avoided. The present invention respects entirely this predecessor-effective, recently developed wound bandaging technology, and recognizes and teaches even further improvements which may be made and employed both in the structure which is used for wound bandaging, and in the associated wound-bandaging methodology which is made available by that structure.

As will be seen, what is contemplated by the present invention are a structure and a methodology involving special, “pliable-application”, dynamic-action wound bandaging. In particular, the bandaging structure and methodology utilize two, cooperative, dynamically behaving mechanisms, that are united in a pliable, anatomically conformable, somewhat fabric-like structure, to act in concert with one another so as to promote rapid wound healing, and to minimize, as much as possible, the “negative” bandaging issues encountered in the past where wound bandaging has itself introduced new problems.

One of these two cooperative mechanisms takes the form of a low-rebound, viscoelastic, acceleration-rate-sensitive cushioning foam material which is applied substantially directly over the area of a wound. This mechanism is “backed up” on its outer side by the other mechanism which features a dynamically performable, pressure-fluid plenum which may be filled and or exhausted with pressure fluid, such as air, or some suitable liquid, to furnish a yieldable dynamic fluid pressure-application “backing” to the mentioned cushioning material. In certain modifications of the structure and methodology of the invention, the pressure-fluid plenum may be equipped with, for example, a pair of appropriately spaced-apart fluid-communication ports which allow pressure fluid, if desired, to be circulated through the bandaging structure and, on the outside of that structure to pass through a heat exchanger whereby heated and or chilled pressure fluid may be made available in the wound-bandaging environment.

While a preferred and best-mode embodiment of, and manner of practicing the invention, essentially involve simply the core operating mechanisms and modalities of the mentioned, acceleration-rate-sensitive cushioning material, and the outer, associated pressure-fluid plenum wherein the pressure of pressure fluid may be adjusted as desired, one should recognize certain variations which may be helpful in the structure and utilization of the invention, such as, for example, (a) the incorporation of a layer, and/or a wrap, of a moisture-wicking material, (b) the provision of an appropriate port structure to enable adjustment of the pressure of pressure fluid in the mentioned pressure-fluid plenum, (c) the employment, where desired, of an external heat-exchange mechanism which may be placed in fluid communication with the pressure-fluid plenum for the supply and exhaust of heated and or cooled fluid, and (d) the construction of the overall bandaging structure in the form of an elongate ribbon which may conveniently and effectively be spirally wound around an arm, a portion of the leg, a knee joint, etc., with, additionally, a optional provision of suitable edge-borne releasable attaching mechanism, such as hook-and-pile releasable attaching mechanism, to accommodate easy spiral winding, fastening, and unfastening, as appropriate.

These and other special features and advantages which are offered and promoted by the structure and the practice of the present invention will become more fully apparent as the description which now follows below is read in conjunction with the accompanying drawings.

### DESCRIPTIONS OF THE DRAWINGS

FIG. 1 is a fragmentary, simplified plan view of a patch-style wound-bandaging structure constructed in accordance with one preferred and best-mode modification of the invention.

FIG. 2 is a simplified, smaller-scale, block/schematic diagram illustrating a version of the structure which is pictured in FIG. 1 connected in a fluid-flow circuit including a fluid pump, a fill/exhaust control valve, and a heat exchanger.

FIG. 3 presents two, enlarged, upper and lower, fragmentary, different-condition cross-sectional views of the bandaging structure of FIG. 1, taken generally along the line 3-3 in FIG. 1, and rotated 90-degrees clockwise. The upper view shows the bandaging structure in a condition with a pressure-inflatable bladder, or plenum, structure which it includes shown in a low-inflation, or non-inflation, state. The lower view illustrates the same bandaging structure with its pressure-inflatable structure in a more inflated, pressurized state. Additionally, a small, left-hand, fragmentary portion of the upper illustration in this figure pictures a modification of the wound-bandaging structure of FIG. 1 wherein a moisture-wicking fabric material is employed only as a single layer disposed against an acceleration-rate-sensitive foam material which is included in the illustrated bandaging structure. In the remainder of FIG. 3, moisture-wicking fabric is more extensively present as a jacket which encloses the other portions, or expanses, of the illustrated bandaging structure. FIG. 3 also, for illustration convenience purposes, shows the presence of a fluid port structure which, strictly speaking, is technically beyond the view-line boundaries of line 3-3 in FIG. 1 where this same port structure is also presented, and thus is technically “out-of-place” in terms of its position as illustrated in FIG. 3.

FIG. 4 is a fragmentary, simplified, smaller-scale (than that employed in FIG. 3) view of another modification of the wound-bandaging structure of the present invention which takes the form of an elongate, somewhat fabric-like ribbon intended, in accordance with appropriate length considerations, to be spirally wound, and edge-to-edge joined as a unified spiral winding, about a subject's limb, such as around the knee. A serpentine, dashed, single arrowheaded line in this figure is intended to illustrate prospective spiral winding.

FIG. 5 is a fragmentary illustration, on a slightly smaller scale than that employed in FIG. 4, of the wound-bandaging structure of FIG. 4 in a condition operatively wound around a knee.

FIG. 6 is similar to FIG. 5, with the exception that a portion of the spiral winding which is pictured in FIG. 6 is broken away to illustrate certain details of in-place implementation.

FIG. 7 is an enlarged, fragmentary detail taken from the area generally embraced by curved arrow 7 in FIG. 6.

### DETAILED DESCRIPTION OF THE INVENTION

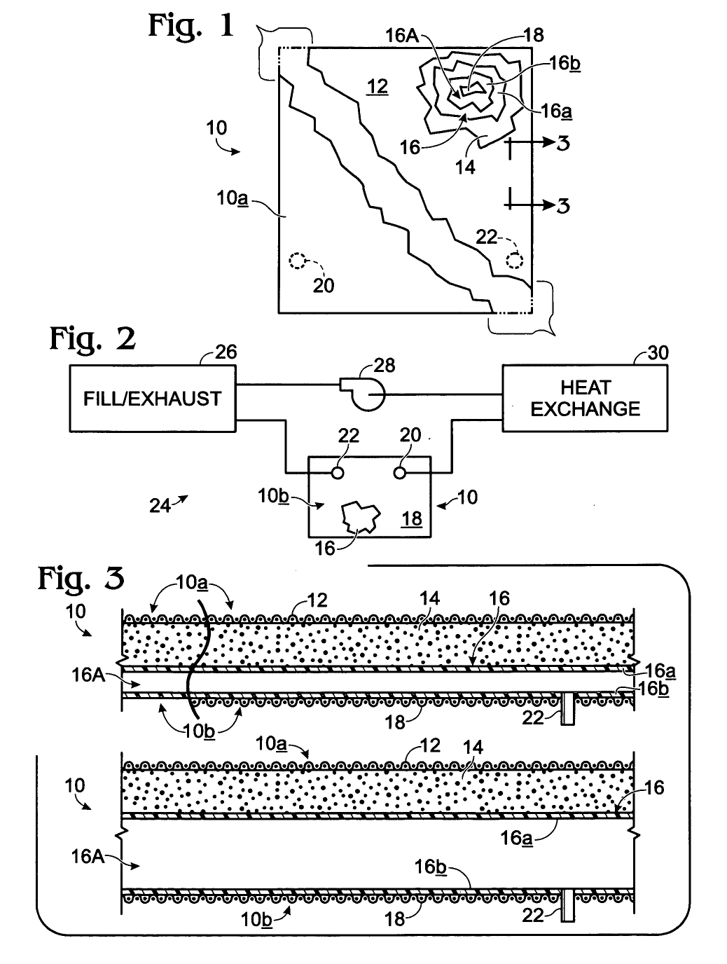

Turning now to the drawings, and referring first of all to FIGS. 1-3, inclusive, indicated generally at 10 is a preferred and best-mode patch-form modification, or embodiment, of a dynamic-action, pliable, anatomically conformable (in a pressure and temperature sense), layered wound-bandaging structure constructed in accordance with the present invention. Structure 10 includes what is referred to herein as an anatomy side 10a and a non-anatomy side 10b. When bandaging structure 10 is in place in relation to a wound area, it substantially completely covers that area, as determined, of course, by one having chosen an appropriate bandaging-structure size—entirely a matter of choice under the control of the user—with anatomy side 10a disposed, effectively, directly adjacent and against the wound area, and non-anatomy side 10b facing outwardly away from that area.

As was mentioned above herein with respect to the descriptions of the several drawings, in FIG. 3, the upper and lower illustrations therein picture structure 10 in two different “internal” conditions, dictated by the pressure-fluid condition, which will shortly be explained, in existence within (in the “interior” of) a pressure-fluid-inflatable structure which is included in the bandaging structure in the form of a bladder, or a plenum. For the most part, however, the structural makeup of the bandaging structure shown in these upper and lower illustrations is the same. Having just said this, we must note that there is one small structural difference which does differentiate the upper and the lower structural views in FIG. 3, and this occurs near the left side of the upper illustration, wherein there appears an evident, irregular, generally vertically deployed fragmentation line, on the respective left and right sides of which are pictured two slightly different internal compositions for structure 10. Thus, and as was also mentioned briefly above in the description of the drawings, FIG. 3 is employed not only to illustrate the structure of FIG. 1 in two different internally-configured operating conditions, but also to illustrate one slightly modified form of the proposed bandaging structure.

It should be pointed out that, while the patch-structure form of bandaging structure 10 as pictured particularly in FIG. 1, and also in FIG. 2, is shown to possess a rectangular, lateral shape, this specific shape is not of any particular consequence. In other words, it should be clearly understood that a patch form of the invention may, if desired, be circular, ovate, or any other perimeter form that is desired.

Describing now the layer organization of bandaging structure 10, and “progressing” in stages through this structure from anatomy side 10a to non-anatomy side 10b, structure 10 includes a layer 12 of a suitable moisture-wicking fabric material, such as the material sold under the trademark Orthowick, a product made by Velcro Laminates, Inc. in Bristol, Ind., having a thickness, perhaps, of up to about 1/16-inches, and next to layer 12, a pliable, low-rebound, viscoelastic, acceleration-rate-sensitive cushioning expanse, or layer, 14 having a thickness lying preferably within the range of about ½-inches to about 1½-inches. In structure 10 as illustrated, expanse 14 has a thickness of ½-inches. The cushioning expanse, which also features anatomical, topographical conformability in relation to temperature and pressure, is formed preferably of a product made by EAR Specialty Composites in Indianapolis, Ind., sold under the trademark Confor® and bearing the product designator Confor-42. Two other Confor® materials which have been found to be very appropriate in certain applications bear the product designators Confor-40 and Confor-45.

Further included in the construction of bandaging structure 10 is an inflatable/deflatable, pressure-fluid bladder, or plenum, 16 having inner and outer sides 16a, 16b, respectively, and also referred to herein as being an adjustable plenum expanse whose interior 16A (see FIG. 3) is adjustable in accordance with the amount and pressure of pressure fluid disposed inside the plenum. The present invention contemplates the either gas (typically air) or some suitable liquid (such as water) may be employed as a pressure fluid. Bladder 16 may be formed of any suitable gas or liquid pressure-fluid-capable material, such as polyurethane, polypropylene, neoprene, urethane, natural, rubber, and others. Under what might be thought of as being nominal conditions for structure 10, where bladder 16 is essentially non-inflated, such a condition generally being shown in the upper illustration of FIG. 3, the overall thickness of structure 10 is about 1-inches. In the lower illustration of FIG. 3, bandaging structure 10 has a thickness of about 1½-inches to about 2-inches.

Continuing a description of the make-up of bandaging structure 10, included therein next to bladder side 16b is another moisture-wicking fabric layer 18 which is essentially made of the same material as that employed in previously described moisture-wicking layer 12.

In FIG. 1, and in the upper and lower illustrations presented in FIG. 3, with the exception of the small, fragmentary, modification portion appearing at the left side of the upper illustration in FIG. 3, moisture-wicking fabric layers 12, 18 are substantially coextensive with expanses 14, 16, and are preferably joined to one another around the perimeter of structure 10 to form an all-around jacketing enclosure, or wrap, fully covering the cushioning and plenum expanses in the structure. The small, fragmentary, modified-structure presentation which is shown at the left side of the upper illustration in FIG. 3 pictures a somewhat changed version of the invention, wherein only a single moisture-wicking fabric layer, namely, layer 12, is employed, with this single layer being disposed on the anatomy side 10a of structure 10.

The different “layer” materials included in structure 10 are preferably surface-bonded to one another at their interfaces by any suitable form of contact adhesive, such as a spray-on contact adhesive.

Further included in bandaging structure 10 is pressure-fluid port structure, here including two spaced-apart fluid ports 20, 22 which are suitably included in the bandaging structure to provide access from its non-anatomy side 10b to the interior 16A of plenum 16. These two ports, in terms of their specific constructions, do not have any special structural features that are associated with the present invention, and, accordingly, may be made in any suitable, conventional fashion. As will be explained, ports 20, 22 enable the selective filling, exhausting and through-flowing of pressure fluid with respect to the interior of plenum 16. The filling of this interior enables, during bandaging use of the invention, the application of, selectively changeable, or fixable, dynamically responsive fluid pressure as a bandaging co-actor with cushioning response 14, which is itself a dynamic-action pressure applier during bandaging. Through-flowing of pressure fluid enables useful heating or cooling heat exchange to take place in certain bandaging applications.

With regard to the presence of fluid port structure, it should also be understood that while the port structure described so far has been illustrated as including a pair of spaced-apart ports, a useful modification of the invention could include port structure having but a single port which enables simply inflation and deflation, i.e., filling and exhausting, of plenum 16 with respect to pressure fluid. Such a single-port structure is useful in applications where the throughflow of fluid is not desired. The bandaging structure of the invention may also, of course, operate in a single-port fashion where two ports are included, but with only one port employed for the flowing of pressure fluid. In all applications, the dynamic, pressure-applying contribution of plenum 16 in the bandaging structure of the invention depends upon the pressure and type of fluid present in the plenum.

Still another possible modification of the invention is one wherein no port structure is included, and where plenum 16 is pre-inflated to a user-selectable, desired level. If one simply imagines FIG. 1 as illustrating no port structure, such a vision of this figure illustrates this further invention embodiment.

Turning attention now to FIG. 2, indicated generally at 24 is a through-flow permitting pressure-fluid circuit which is illustrated as including bandaging structure 10, fluid ports 22, 24, plenum 16, a conventional, adjustable fill/exhaust valve 26, an adjustable, pressure-fluid (air, or liquid) pump 28, and a conventional heat exchanger 30. Under a circumstance where the bandaging structure includes but a single fluid port, then, heat exchanger 30 would be omitted, and all that would be connected, and/or connectable, as desired to just a single fluid ports would be an appropriate fill/exhaust valve 26 and an appropriate pump 28. Obviously, pump 28 is suitably connected to the atmosphere where air is to be employed in plenum 16, or to an appropriate reservoir of other gas or a liquid. Valve 26 and pump 28 are, of course, appropriately operable to control the filling, exhausting, and through-flowing, as desired, of pressure fluid with respect to the fluid port structure and the interior 16A of plenum 16.

The just-described patch form of the invention, when in use, may be held in place over a wound area in the manner described in the above-referenced '375 patent.

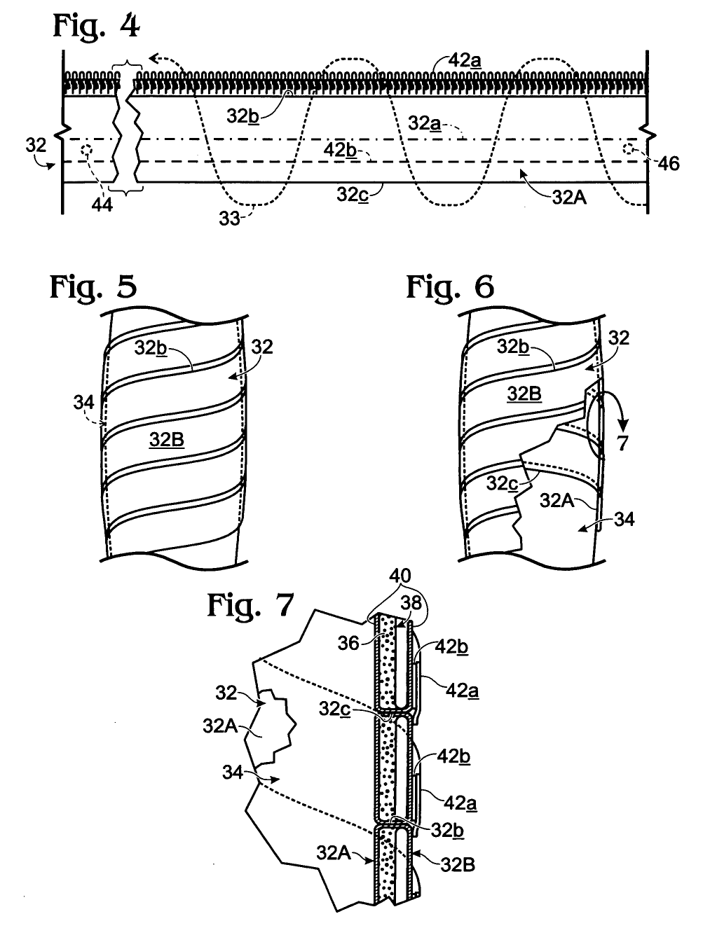

FIGS. 4-7, inclusive, illustrate at 32 another modified form of wound-bandaging structure which is constructed in accordance with preferred, and best-mode, alternative embodiment of the present invention. Bandaging structure 32 is configured as an elongate, pliable, and generally fabric-like ribbon having a long axis shown by a dash-dot line 32a in FIG. 4, and a pair of spaced, lateral edges 32b and 32c. This elongate, ribbon form of the invention is adapted especially to be wrapped in the form of a spiral winding around a wound in a limb, such as around a surgical wound in a person's knee, like the fragmentary portion of a knee which is shown generally at 34 in FIGS. 5-7, inclusive. Such spiral winding is suggested in FIG. 4 by a dashed, serpentine line 33. Ribbon 32 further includes an anatomy side 34A, and a non-anatomy side 34B (the latter being seen only in FIGS. 5-7, inclusive).

With reference made especially to FIG. 7, bandaging structure 32 includes an elongate cushioning expanse 36, and an elongate pressure-fluid plenum 38, which, in structure and functionality, are like previously described cushioning and plenum expanses 14, 16, respectively. Structure 32 also includes an “expanse-jacketing” wrap 40 of moisture-wicking fabric, like the wrap formed of previously identified moisture wicking fabric layers 12, 18.

Suitably fastened to the opposite lateral edges of bandaging structure 32 are two, edge-to-edge fastening components 42a, 42b which are associated, respectively, with ribbon edges 32b, 32c. These two components of what is referred to herein as edge-to-edge fastening structure preferably take the form of appropriate components of elongate bands of complementarily releasably interengageable, hook-and-pile material, such as the material well known and sold under the trademark Velcro® trademark. Band 42a is fastened to ribbon edge 32b in such a fashion that it effectively forms an outward, lateral extension of this edge. What may be thought of as the “working side”, or face, of band 42a faces in the “direction” of anatomy side 32A of structure 32 (toward the viewer in FIG. 4). Band 42b is fastened just immediately inwardly of ribbon edge 32c, on the non-anatomy side of the ribbon, and has its “working side” facing in the “direction” of non-anatomy side 32B (i.e., away from the viewer in FIG. 4).

Additionally included in the bandaging structure pictured in FIGS. 4-7, inclusive, is dual-port pressure-fluid port structure like that which is illustrated in bandaging structure 10, including spaced-apart fluid ports 44, 46 which are seen in FIG. 4. Preferably, these ports are located adjacent opposite ends of whatever length (an entirely user selectable parameter) has been chosen for ribbon structure 32.

As can be seen, and as has been mentioned, ribbon bandaging structure 32 is designed for convenient and effective spiral winding or wrapping around an anatomical limb, such as the leg, and as has already been mentioned, structure 32 is so pictured in FIGS. 5-7 wound around knee 34. The described edge-to-edge fastening structure enables quick and easy releasable edge fastening in such a winding arrangement, and this fastening condition is clearly pictured in FIG. 7.

The ribbon, spiral-winding modification of the present invention is obviously a very convenient structure to employ in many wound-bandaging conditions, and, as was mentioned earlier, the length of the structure is entirely user selectable. This structure enables convenient and full application of wound bandaging to a wound area, and especially to a relatively widely distributed wound area.

In all of the versions of the present invention, the dual, pliable, anatomically conformable dynamic-action capability of the proposed structure, utilizing both acceleration-rate-rate-sensitive cushioning structure, and an associated and integrated “backing” of a pressure-fluid plenum, offers a great deal of versatility in bandaging applications, and allows for very fine and effective control over wound-bandaging pressure application.

The structure of the present invention accurately and very successfully addresses all of the above-mentioned concerns that involve otherwise conventional wound-bandaging structures and methodologies. The acceleration-rate-sensitive foam structure, under applied pressure against the anatomy, responds to this pressure, and to body temperature, to conform very precisely to the topographic anatomical features in the bandaged area. Extremely even pressure is applied throughout an entire wound-bandaged region, and anatomical movement, including blood pulsatile behavior, is immediately followed by the structure of the invention in a manner which tends to maintain applied bandaging pressure substantially constant and under all circumstances, and evenly deployed over a wound area. Anatomical movements, as well as any anatomical protrusions, such as below-the-skin bone structure, will not produce high pressure points, and will not introduce, adjacent potential high-pressure points, any uneven low-pressure points. Wound healing observed in test applications of the structure and methodology of the invention has been seen to take place rapidly, and without the introduction of any additional injuries such as those mentioned earlier herein.

From one methodologic point of view, and in general terms, what is proposed uniquely by the present invention is a dynamic bandaging practice which includes the steps of (a) applying to the surface of the anatomy, over a wound area, the anatomy side of a first, dynamic bandaging structure which takes the form of a low-rebound, viscoelastic, acceleration-rate-sensitive cushioning material, (b) by such applying, furnishing the wound area with a dynamic, low-rebound, viscoelastic, acceleration-rate-sensitive, anatomically conforming, pressure-applying behavior, and (c) utilizing a second, dynamic bandaging structure which takes the form of a controlled, contained, pressure-fluid instrumentality, and which is disposed adjacent and against the first bandaging structure's non-anatomy side, applying to that non-anatomy side, and thence through the first bandaging structure to the wound area, dynamic, controlled, contained, pressure-fluid, pressure-distributing action.

On a final point, there are certain situations, such as certain pre-surgery situations (as, for example, a pre-foot-surgery situation), with respect to which the bandaging structure and methodology of the present invention may be useful to pre-create a fluid-diminished region in anticipation of surgery in that region. Accordingly, the concept of wound bandaging herein is intended to be applicable in such a situation, and the terminology “wound region”, etc., as employed in the description and claiming of the present invention may be considered, in this context, to refer to a pre-surgical wound area.

Accordingly, while preferred and best-mode embodiments of the structure, and of the practice methodology, of the present invention have been illustrated and described herein, and certain variations and modifications variously pictured, described and suggested, it is appreciated that other variations and modifications may be made without departing from the spirit of the invention.

I claim:

1. A dynamic-action, pliable, anatomically conformable bandaging structure for application to an anatomical wound in a portion of the body comprising
a pliable, low-rebound, viscoelastic, acceleration-rate-sensitive cushioning expanse placeable confrontingly adjacent such a wound, and
operatively associated and structurally united with said expanse, adjustable, pressure-fluid-inflatable structure.

2. Layered, dynamic-action, pliably, anatomically conformable wound-bandaging structure having opposite, anatomy and non-anatomy sides comprising
a low-rebound, viscoelastic, acceleration-rate-sensitive cushioning expanse disposed in said bandaging structure toward the bandaging structure's said anatomy side, and
a pressure-fluid-containing plenum expanse operatively associated with said cushioning expanse, and disposed in said bandaging structure immediately adjacent said plenum expanse and toward the bandaging structure's said the non-anatomy side.

3. The wound-bandaging structure of claim 2, wherein said two expanses are substantially coextensive.

4. The wound-bandaging structure of claim 3 which takes the form of an elongate ribbon which is windable about a selected portion of the anatomy.

5. The wound-bandaging structure of claim 2, wherein said plenum expanse has an interior, and the wound-bandaging structure includes fluid port structure which is in fluid communication with said interior, said fluid port structure accommodating the selective supplying, exhausting, and sealing of pressure fluid, respectively, into, out of, and within said interior.

6. The wound-bandaging structure of claim 5 which takes the form of an elongate ribbon which is windable about a selected portion of the anatomy.

7. The wound-bandaging structure of claim 2, wherein said cushioning expanse possesses an anatomy side, and which further includes a layer of moisture-wicking fabric disposed adjacent and generally spanning said cushioning expanse's said anatomy side.

8. The wound-bandaging structure of claim 2 which further includes a layer of moisture-wicking fabric jacketing said cushioning and plenum expanses.

9. The wound-bandaging structure of claim 2 which takes the form of an elongate ribbon which is windable about a selected portion of the anatomy.

10. The wound-bandaging structure of claim 9, wherein said cushioning expanse possesses an anatomy side, and which further includes a layer of moisture-wicking fabric disposed adjacent and generally spanning said cushioning expanse's said anatomy side.

11. The wound-bandaging structure of claim 9, which further includes a layer of moisture-wicking fabric jacketing said cushioning and plenum expanses.

12. The wound-bandaging structure of claim 9, wherein said ribbon possesses spaced, elongate, lateral edges, is windable in a spiral-wrap manner about such a portion of the anatomy, and includes elongate, complementarily releasably interengageable, edge-to-edge fastening structure distributed along said lateral edges.

13. The wound-bandaging structure of claim 12, wherein said fastening structure takes the form of hook-and-pile material.

14. A dynamic-action, pliable, anatomically conformable bandaging structure for application to an anatomical wound in a portion of the body comprising
a pliable low-rebound, viscoelastic, acceleration-rate-sensitive cushioning expanse placeable confrontingly adjacent such a wound, and
operatively associated and structurally united with said expanse, pressure-fluid-inflated structure.

15. Dynamic bandaging methodology comprising
applying to the surface of the anatomy over a wound area the anatomy side of a first, dynamic bandaging structure which takes the form of a low-rebound, viscoelastic, acceleration-rate-sensitive cushioning material, and which also possesses a non-anatomy side,
by said applying, furnishing the wound area with a dynamic, low-rebound, viscoelastic, acceleration-rate-sensitive pressure-applying behavior, and
utilizing a second, dynamic bandaging structure which takes the form of a controlled, contained, pressure-fluid instrumentality, and which is disposed adjacent and against the first bandaging structure's mentioned non-anatomy side, applying to that non-anatomy side, and thence through the first bandaging structure to the wound area, dynamic, controlled, contained pressure-fluid action.

16. The bandaging methodology of claim that 15, wherein the first and second bandaging structures are integrated in the form of an elongate bandaging ribbon, and said applying is performed by winding the bandaging ribbon about a portion of the anatomy in the vicinity of the wound.

17. The bandaging methodology of claim that 16 wherein said winding is carried out in a spiral-wrapped, edge-to-edge releasably fastened manner.
